(19) United States
(12) Patent Application Publication
(10) Pub. No.: US 2008/0167631 A1
(43) Pub. Date: Jul. 10, 2008

(54) Subatmospheric pressure dressing

(76) Inventor: Steven E. Greer, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/977,814

(22) Filed: Oct. 26, 2007

Related U.S. Application Data
(60) Provisional application No. 60/854,600, filed on Oct. 26, 2006.

(51) Int. Cl. A61F 13/02

(52) U.S. Cl. 604/290; 602/53

(57) ABSTRACT

A subatmospheric pressure dressing can be readily applied by a single person and includes a flexible base which supports a gas permeable screen. A flexible sheet encloses the base and screen. The screen is connected with a source of low pressure (vacuum) by a suction tube. Transmittal of low fluid pressure causes the flexible sheet to be drawn into close contact with the screen to indicate that suction has been established. If a leak develops, the flexible sheet is no longer drawn to the screen to provide a clear indication of the leak.

### BACKGROUND OF THE INVENTION

The present invention relates to a method and apparatus for use in treating a wound. More specifically, the present invention relates to the application of subatmospheric pressure to a wound.

It has been suggested that wounds may be treated by the application of relatively low pressure to the wound. This has involved the use of a porous wound screen which may be formed of an open cell polymer foam. The wound screen is connected with a source of vacuum by a suction tube. The wound screen is enclosed by a flexible polymer sheet which is secured to a patient's body by adhesive. One known apparatus having such a construction is disclosed in U.S. Pat. No. 5,636,643.

The use of this known device to apply subatmospheric pressure or suction to a wound is complicated by the fact that the adhesive is connected with the flexible polymer sheet. When the flexible polymer sheet is to be placed over the wound screen, the adhesive tends to attach to portions of the flexible polymer sheet and/or the wound screen. In order to position the flexible polymer sheet over the wound screen and to connect the sheet in a desired orientation relative to a patient's body, two people are usually required. One of these two people holds the wound screen in place and holds a portion of the flexible polymer sheet. The other person positions the polymer sheet relative to the patient.

### SUMMARY OF THE INVENTION

The present invention provides a new and improved subatmospheric pressure dressing which can be readily applied to a patient by a single person. The subatmospheric pressure dressing may include a base formed of a hydrocolloid or other material. This base is flexible so that it can be deflected to the configuration of a patient's body. However, the base has sufficient rigidity to provide support for a gas permeable screen which may be formed of an open cell polymeric foam or other porous material.

The screen and at least a portion of the base are enclosed by a flexible sheet. The sheet is connected with the base by a fluid tight seal. An adhesive layer on the base enables the base to be hermetically sealed to the patient's body with the screen directly over a wound.

The screen is connected with a source of low pressure (vacuum) by a suction tube. During use of the subatmospheric pressure dressing, low pressure transmitted through the suction tube draws edema and/or other fluids away from the wound.

When the subatmospheric pressure dressing is to be applied, it is merely necessary to remove a protective layer from the adhesive on the base to expose the adhesive. The entire dressing may then be accurately positioned on a patient's body by one person. The suction tube is then connected with a source of vacuum.

The transmittal of low fluid pressure, that is, subatmospheric pressure, to the screen causes the flexible sheet to be drawn into close contact with the screen, thereby indicating that suction has been established. If for any unforeseen reason a leak should develop between the base and the patient's body, the flexible sheet will no longer be drawn to the screen. This provides a clear indication to personnel attending to the patient that suction has been lost.

### BRIEF DESCRIPTION OF THE DRAWINGS

The foregoing and other features of the invention will become more apparent upon consideration of the following description taken in connection with the accompanying drawings wherein:

FIG. 1. is a schematic pictorial illustration of a subatmospheric pressure dressing constructed in accordance with the present invention prior to positioning of the dressing on a patient's body; and

FIG. 2. is a fragmentary sectional view illustrating the manner in which the subatmospheric pressure dressing of FIG. 1 is positioned on a patient's body.

### DESCRIPTION OF ONE SPECIFIC PREFERRED EMBODIMENT OF THE INVENTION

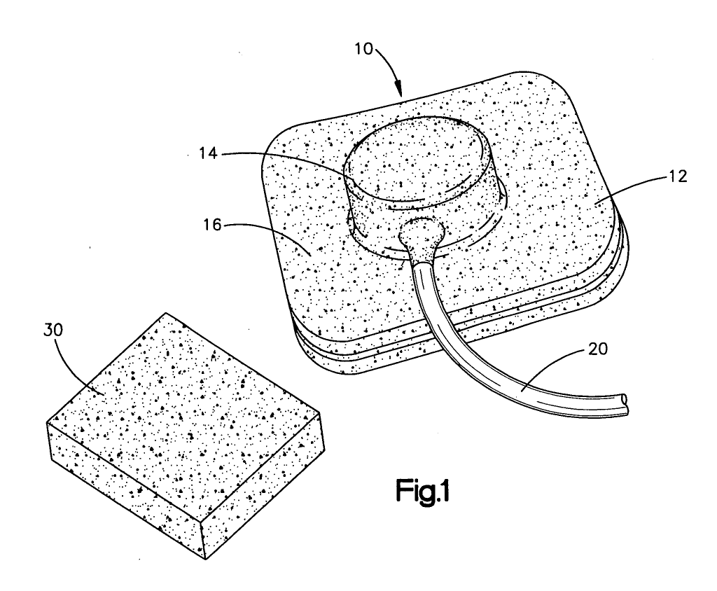

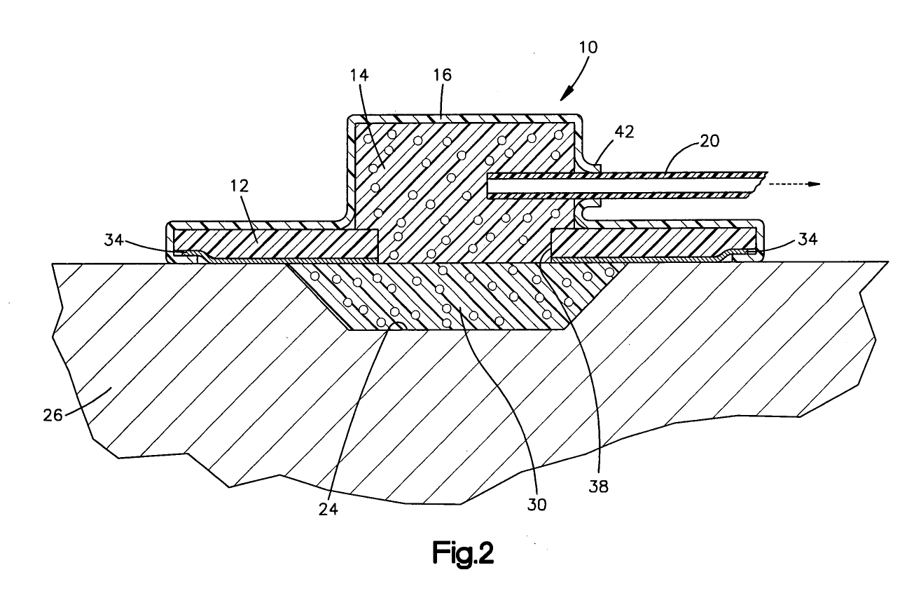

A subatmospheric pressure dressing (SPD) 10 constructed in accordance with the present invention is illustrated in FIGS. 1 and 2. The subatmospheric pressure dressing 10 includes a base 12 and a porous screen 14. The porous screen is enclosed by a flexible, fluid impervious sheet 16. In addition to extending around the exterior of the porous screen 14, the flexible sheet 16 extends across the upper side of the base 12 and is sealingly secured to the base 12.

A suction tube 20 extends through the sheet 16 and into the porous screen 14. Fluid tight seals are formed between the suction tube 20, sheet 16 and base 12. These fluid tight seals may be formed by a suitable adhesive.

When the subatmospheric pressure dressing 10 is to be positioned in association with a wound 24 (FIG. 2) in a patient's body 26, an initial dressing 30 is placed in engagement with the wound. The initial dressing is formed of a fluid permeable material which is cut to fit the wound 24. The initial dressing may be an open cell polymeric foam or a gauze.

Once the initial dressing 30 has been positioned on the wound 24, the subatmospheric pressure dressing 10 is positioned on the patient's body. To position the dressing 10 on the patient's body, a protective paper covering is removed from a layer 34 of adhesive on the lower side of the base 12. Once the protective covering has been removed from the layer 34 (FIG. 2) of adhesive, the dressing 10 is manually positioned over the wound 24 with the porous screen 14 centered relative to the wound.

The layer 34 of adhesive extends across the bottom surface of the base 12. The layer 34 of adhesive is firmly pressed against the patient's body 26. This results in the adhesive 34 forming a fluid tight seal (hermetic) between the base 12 and skin on the patient's body 26 around to the wound 24. In the embodiment of the invention illustrated in FIG. 2, the layer 34 of adhesive also seals the sheet of polymeric material to the base 12.

After the paper covering has been removed from the layer 34 of adhesive, the entire dressing 10 can be readily positioned on the patient's body with only one hand. Thus, a nurse or other person may position the entire dressing 10 relative to the wound 12 with one hand and press the base 12 and layer 34 of adhesive firmly against the patient's body with the other hand. This enables the dressing to be installed, with little or no difficulty, by a single person.

The base 12 consists of a flexible, polyurethane upper (as viewed in FIGS. 1 and 2) foam layer and a hydrocolloid inner or lower layer. Although the base 12 may be formed of many different materials, it is believed that the base 12 may advantageously be formed of “DuoDERM” (trademark) which is commercially available from E.R. Squibb and Sons, Inc. and is distributed by drug stores and medical supply outlets. Of course, the base 12 may be formed of a different material if desired. However, the base 12 should be flexible so as to be readily deflected to conform to the configuration of a patient's body. The base 12 should also have sufficient rigidity so that it will retain its configuration when the dressing is held as a unit by one hand of a person applying the dressing to a patient's body.

The porous screen 14 is formed of an open cell polymeric foam. However, it should be understood that the screen 14 may be formed of other materials if desired. Thus, the screen 14 may be formed of a fluid permeable material, such as a felt or gauze.

In the embodiment of the invention illustrated in FIG. 2, a circular central opening 38 is formed in the base 12 (FIG. 2). The cylindrical screen 14 extends into the central opening 38 in the base 12. The screen 14 is secured to the base 12 by a suitable adhesive which is positioned on an interior surface of the central opening 38. Additional adhesive to secure the screen 14 to the base 12 may be positioned on an upper side surface of the base 12.

If desired, the screen 14 may span the central opening 38 without extending through the central opening in the manner illustrated schematically in FIG. 2. If the screen 14 spans the central opening 38 in the base 12, the screen may be secured to the upper side surface of the base with a suitable adhesive.

The flexible sheet 16 encloses the screen 14 and the upper side and periphery of the base 12. The sheet 16 is secured to the lower side and periphery of the base 12 by adhesive including the layer 34 of adhesive. In addition, the sheet 16 may be secured to the upper (as viewed in FIG. 2) side of the base 12 by adhesive. Thus, a band of adhesive may be provided along the upper side of the base 12 adjacent to the periphery of the base. In any case, the fluid impermeable flexible sheet 16 is sealed to the base 12. Gas cannot leak between the sheet 16 and the base 12 and cannot pass through the sheet.

The sheet 16 is advantageously formed of a clear transparent polymeric material which is fluid impenetrable. By having the sheet 16 formed of a clear transparent polymeric material, a nurse or other personnel can view the screen 14 through the sheet. Regardless of whether or not the sheet 16 is formed of a clear plastic material, the sheet 16 is sufficiently flexible so that suction (subatmospheric pressure) transmitted through the tube 20 is effective to draw the sheet 16 tightly against the exterior of the screen 14 and against the base 12. This tight engagement of the sheet 16 with the outside of the screen 14 and base 12 results from the relatively high atmospheric pressure to which the exterior surface of the sheet is exposed and the relatively low, subatmospheric, pressure in the interior of the porous, gas permeable screen 14.

The suction tube 20 extends through a passage in the sheet 16 and into a cylindrical opening formed in the porous open cell foam of the screen 14. A portion 42 (FIG. 2) of the sheet 16 extends around the suction tube 20. The sheet 16 is secured to the suction tube with a gas tight seal by a suitable adhesive. As was previously mentioned, the flexible sheet 16 is secured to the base 12 with a gas light seal by adhesive which may include the layer 34 of adhesive. Thus, a layer of adhesive may be provided between the bottom surface and/or top surface of the base 12 and the sheet 16. The layer or layers of adhesive would extend completely around the periphery of the base 12.

It is contemplated that the portion of the suction tube 20 extending into the screen 14 may be connected with the screen by an adhesive coating on at least a portion of the exterior surface of the suction tube which is received in the cylindrical opening formed in the screen 14. However, there is no adhesive at the end of the suction tube which is disposed in the screen 14. This enables fluid to be drawn into the suction tube 20 from the screen 14.

Once the dressing 10 has been secured over the wound 24 in a patient's body in the manner illustrated in FIG. 2, the suction tube 20 is connected with a suitable source of vacuum, such as a bed side suction pump or hospital wall suction. If desired, a small portable suction pump may be connected with the dressing 10 by the suction tube 20. This results in a subatmospheric microenvironment being created over the wound 24.

The subatmospheric environment over the wound 24 draws edema and other fluids away from the wound. In addition, the suction is effective to increase local blood flow, decrease bacterial load and promote granulation of tissue, all of which is beneficial to wound healing. Since the exterior surface of the sheet 16 and screen 14 are pressed firmly against the initial dressing 30 and the wound 24 by atmospheric pressure, a force is applied to the wound to promote effective skin graph over irregular surfaces. In addition, the subatmospheric pressure in the gas permeable initial dressing 30 and the gas permeable screen 14 promotes a flow of edema from the wound 24 due to the relatively high fluid pressure in the patient's body 26.

### CONCLUSION

The present invention provides a new and improved subatmospheric pressure dressing 10 which can be readily applied to a patient by a single person. The subatmospheric pressure dressing 10 may include a base 12 formed of a hydrocolloid or other material. This base 12 is flexible so that it can be deflected to the configuration of a patient's body. However, the base 12 has sufficient rigidity to provide support for a gas permeable screen 14 which may be formed of an open cell polymeric foam or other porous material.

The screen 14 and at least a portion of the base 12 are enclosed by a flexible sheet 16. The sheet 16 is connected with the base 12 by a fluid tight seal. An adhesive layer 34 on the base 12 enables the base to be hermetically sealed to the patient's body 26 with the screen 14 directly over a wound 24.

The screen 14 is connected with a source of low pressure (vacuum) by a suction tube 20. During use of the subatmospheric pressure dressing 10, low pressure transmitted through the suction tube 20 draws edema and/or other fluids away from the wound 24.

When the subatmospheric pressure dressing 10 is to be applied, it is merely necessary to remove a protective layer from the adhesive 34 on the base 12 to expose the adhesive. The entire dressing 10 may then be accurately positioned on a patient's body 26 by one person. The suction tube 20 is then connected with a source of vacuum.

The transmittal of low fluid pressure, that is, subatmospheric pressure, to the screen 14 causes the flexible sheet 16 to be drawn into close contact with the screen, thereby indicating that suction has been established. If for any unforeseen reason a leak should develop between the base 12 and the patient's body 26, the flexible sheet 16 will no longer be drawn to the screen 14. This provides a clear indication to personnel attending to the patient that suction has been lost.

Having described the invention, the following is claimed:

1. A subatmospheric pressure dressing assembly comprising a base, a gas permeable screen connected with said base, a suction tube extending into said screen, a flexible gas impermeable layer overlying said screen and at least a portion of said base with a fluid tight seal between said flexible layer and said base, and a layer of adhesive disposed on a lower side of said base to secure said base to a patient's body and form a fluid tight seal with the patient's body.

2. A method of installing a subatmospheric pressure dressing assembly over a wound on a patient's body, said method comprising the steps of positioning a porous screen in the subatmospheric pressure dressing assembly relative to the wound on the patient's body, pressing adhesive on a lower side of a base of the pressure dressing assembly against the patient's body to form a fluid tight seal with the patient's body around the wound, and connecting a suction tube extending from the subatmospheric pressure dressing assembly with a source of subatmospheric pressure.
